(19) United States
(12) Patent Application Publication
(10) Pub. No.: US 2009/0203977 A1
(43) Pub. Date: Aug. 13, 2009

(54) METHOD OF SCREENING FOR CANCER USING PARAMETERS OBTAINED BY THE DETECTION OF EARLY INCREASE IN MICROVASCULAR BLOOD CONTENT

(76) Inventors: Vadim Backman, Chicago, IL (US); Hemant Roy, Highland Park, IL (US); Andrew Gomes, Chicago, IL (US); Sarah Ruderman, Evanston, IL (US); Jeremy Rogers, Chicago, IL (US)

Correspondence Address: PILLSBURY WINTHROP SHAW PITTMAN LLP, P.O. BOX 10500, MCLEAN, VA, 22102, US

(21) Appl. No.: 12/350,955

(22) Filed: Jan. 8, 2009

Related U.S. Application Data
(63) Continuation-in-part of application No. 11/604,653, filed on Nov. 27, 2006.
Application No. 11/604,653 is a continuation-in-part of application No. 11/604,659, filed on Nov. 27, 2006.
Application No. 11/604,659 is a continuation-in-part of application No. 11/261,452, filed on Oct. 27, 2005.
(60) Provisional application No. 60/801,947, filed on May 19, 2006.

(51) Int. Cl. A61B 5/1459

(52) U.S. Cl. 600/325; 600/342

(57) ABSTRACT

The present invention, in one aspect, relates to screening test for tumors or lesions using what is referred to as “Early Increase in microvascular Blood Supply” (EIBS) that exists in tissues that are close to, but are not themselves, the abnormal tissue and in tissues that precede the development of such lesions or tumors. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

### PRIORITY CLAIM

This application is a continuation in part and claims priority to related to co-pending U.S. patent application Ser. No. 11/604,653 filed Nov. 27, 2006, entitled “Method of Recognizing Abnormal Tissue Using the Detection of Early Increase in Microvascular Blood Content”, the disclosure of which is incorporated in its entirety by reference, which application claims priority to U.S. Application No. 60/801,947 entitled “Guide-To-Colonoscopy By Optical Detection Of Colonic Micro-Circulation And Applications Of Same”, which was filed on May 19, 2006, the contents of which are expressly incorporated by reference herein.

This application is also a continuation in part and claims priority to related to co-pending U.S. patent application Ser. No. 11/604,659 filed Nov. 27, 2006 and entitled “Apparatus For Recognizing Abnormal Tissue Using The Detection Of Early Increase In Microvascular Blood Content,” the contents of which are expressly incorporated by reference herein.

This application is a continuation-in-part and claims priority to co-pending U.S. patent application Ser. No. 11/261,452 entitled “Multi-Dimensional Elastic Light Scattering”, filed Oct. 27, 2005, the contents of which are incorporated in its entirety herein by reference.

Some references, which may include patents, patent applications and various publications, are cited and discussed in the description herein. The citation and/or discussion of such references is provided merely to clarify the description of the present invention and is not an admission that any such reference is “prior art.” All references cited and discussed in this specification are incorporated herein by reference in their entireties and to the same extent as if each reference was individually incorporated by reference.

### STATEMENT AS TO RIGHTS UNDER FEDERALLY-SPONSORED RESEARCH

These inventions were made with Government support under Grant No. R01CA109861 awarded by the National Institutes of Health of the United States. The United States Government has certain rights in the invention.

### FIELD OF THE INVENTIONS

The present inventions relate generally to light scattering and absorption, and in particular to methods of recognizing possibly abnormal living tissue using a detected early increase in microvascular blood supply and corresponding applications including in vivo tumor imaging, screening, detecting and treatment, and, in particular, “Early Increase in microvascular Blood Supply” (EIBS) that exists in tissues that are close to, but are not themselves, the lesion or tumor and in tissues that precede the development of such lesions or tumors.

### BACKGROUND

There are various techniques known for determining abnormality in tissues. Of these techniques, those that are most relevant to the present invention are techniques in which there is detected an increase in blood within tissue that is abnormal. While such techniques have advantages in and of themselves as compared to other methods, they require testing of the abnormal tissue itself, which may be difficult to detect. Further, such methods are usable only after the abnormality is sufficiently large, such as a cancerous tissue.

Detecting cancer tissue in colons is one specific area where research continues. Colonoscopy has the potential of reducing colorectal cancer (CRC) occurrence by ˜90% through the identification and interdiction of the precursor lesion, the adenomatous polyp. However, CRC remains the second leading cause of cancer deaths in the United States with an anticipated 148,810 new cases in 2008. The major reason why existing CRC screening strategy is not adequate is that according to existing recommendations, every patient over the age of 50 is considered at risk for CRC and is a candidate for colonoscopic surveillance to be performed at least every 10 years. However, screening the entire eligible population (>90 million Americans over age 50) through colonoscopy is practically impossible for a variety of reasons including expense, patient reluctance, complication rate, and insufficient number of endoscopists. Indeed, currently only less than 20% of the population undergoes colonoscopy. Further compounding this fact is that the vast majority of colonoscopies are negative. For instance, ˜70-80% of patients do not harbor any neoplastic lesions on colonoscopy, Moreover, a vast majority of these adenomas will never develop into colon cancer. For the clinically/biologically significant neoplasia (advanced adenomas) the yield is only ˜5%.

Accordingly, the present invention provides a variety of advantageous optical techniques for assisting in the detection of abnormal tissue, particularly a screening test for colons, using optical measurements, early in the development of the abnormal tissues themselves.

### SUMMARY

The present inventions, in one aspect, relate to a method for screening for tumors or lesions in the human colon using what is referred to as “Early Increase in microvascular Blood Supply” (EIBS) that exists in tissues that are close to, but are not themselves, the abnormal tissue and in tissues that precede the development of such lesions or tumors. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

In a particular embodiment, the screening includes obtaining EIBS measurements and using those measurements to obtain an estimated blood vessel diameter, also known as PLS, and an estimated oxygenated hemoglobin. One or preferably both of the estimated blood vessel diameter and the estimated oxygenated hemoglobin can be used with a prediction rule to screen for colon cancer.

### BRIEF DESCRIPTION OF THE DRAWINGS

These and other aspects and features of the present inventions will become apparent to those of ordinary skill in the art upon review of the following description of specific embodiments of the invention in conjunction with the accompanying figures, wherein:

FIGS. 1(a), (b) and (c) illustrate graphs of supporting data for OHb concentration, packaging length scale (PLS), and normalized packaging length scale, respectively.

FIGS. 2(a) and (b) show concentration of OHb and tissue oxygenation relative to probe tissue contact.

### DETAILED DESCRIPTION OF THE PREFERRED EMBODIMENTS

The present inventions are more particularly described in the following examples that are intended as illustrative only since numerous modifications and variations therein will be apparent to those skilled in the art. Various embodiments of the invention are now described in detail. Referring to the drawings, like numbers indicate like components throughout the views, As used in the description herein and throughout the claims that follow, the meaning of “a”, “an”, and “the” includes plural reference unless the context clearly dictates otherwise Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, not is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The present inventions, in one aspect, relate to methods for examining a target for tumors or lesions using what is referred to as “Early Increase in microvascular Blood Supply” (EIBS) that exists in tissues that are close to, but are not themselves, the lesion or tumor. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

A particular application described herein is for detection of such lesions in colonic mucosa in early colorectal cancer (“CRC”), but other applications are described as well.

The target is a sample related to a living subject such as a human being or animal. The sample is a part of the living subject such that the sample is a biological sample, wherein the biological sample may have tissue developing a cancerous disease.

The neoplastic disease is a process that leads to a tumor or lesion, wherein the tumor or lesion is an abnormal living tissue (either premalignant or cancerous), such as a colon cancer, an adenomatous polyp of the colon, or other cancers.

The measuring step is performed in vivo. The measuring step may further comprise the step of acquiring an image of the target. The image, obtained at the time of detection, can be used to later analyze the extent of the tumor, as well as its location. In use, the probe is inserted into the distal colon for analysis of rectal mucosa, thus provides a mechanism to assess a patient's risk of developing colon cancer without the need for colonoscopy, and also without the need for colon purging when using the probe. Measuring of blood content using interacted light, which can include scattering as well as other optical methods, can include insertion of a probe for in-vivo usages in which blood content and/or flow is measured in tissue of a solid organ. In one embodiment, the method comprises projecting a beam of light to a target that has tissues with blood circulation therein. Light scattered from the target is then measured, and blood supply information related to the target is obtained. The obtained blood supply information comprises data related to blood oxygenation and blood vessel size known as PLS and described herein, which data is then used for screening for colon cancer.

Without intent to limit the scope, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action. There optical measurement techniques that can be used to obtain the data required in order to obtain the blood oxygenation (OHb) and blood vessel size (PLS) are described in the EIBS-related patent applications incorporated by reference above that describe other optical probes and systems that are discussed in the context of detection of EIBS. It is also noted that further EIBS optical probes that can be used for the colon cancer screening discussed herein are described in U.S. Provisional Patent Application Ser. No. 61/143,407, filed Jan. 8, 2009, entitled “Probe Apparatus For Recognizing Abnormal Tissue” which application bears Attorney Reference 042652-0376945, which application is expressly incorporated by reference herein.

The screening technique described herein can also be used in screening for colon cancer. Specifically, this screening is based upon noted observations including that 1) EIBS occurs very early in the process of colon carcinogenesis; 2) EIBS is detectable outside of a neoplastic lesion, such as a colonic adenoma, in endoscopically and histologically normal-appearing (uninvolved) mucosa (i.e. marker of the field effect). One particular parameter obtained from EIBS, as noted above, is the increase of total hemoglobin (Hb) concentration in uninvolved mucosa, which was observed within the same colonic segment (i.e. within that same ⅓ of the colon) where the adenoma is located; 3) Spectroscopic measurements can be used to measure both oxygenated Hb (OHb) concentration in the mucosa and effective blood vessel size (aka. Hb packaging length scale, referred to also as “PLS”). 4) OHb is increased in the mucosa outside of an adenoma in the same segment; 5) PLS is a marker of the field effect. Decrease in PLS (reduction in the average blood vessel size) was observed in the distal colon (rectum) in patients with proximal advanced adenomas. 6) Increase in OHb was observed in the distal colon in patients with proximal advanced adenomas. The effect was particularly pronounced in women.

Both the PLS and the increase in OHb have now been found to be detectable at distances from an adenoma that allow for detection and estimation of one or both of these parameters at one end of the colon (typically the rectum, also referred to as distal colon) as an indicator of whether there exists abnormal tissue anywhere within the entire colon.

Both PLS and OHb effects, as with EIBS as explained hereinbefore, are only be observed if sufficiently shallow tissue was probed, typically ˜100-200 microns below tissue surface consistent with the depth of the mucosa. Thus, these aspects of EIBS develop primarily in the mucosa.

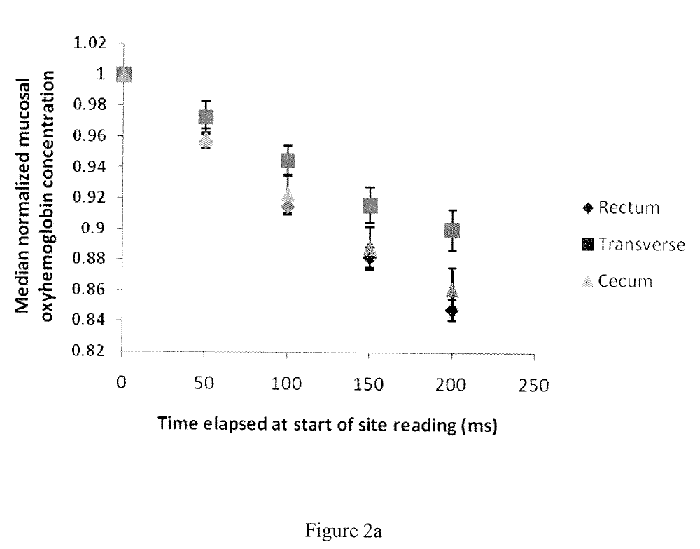

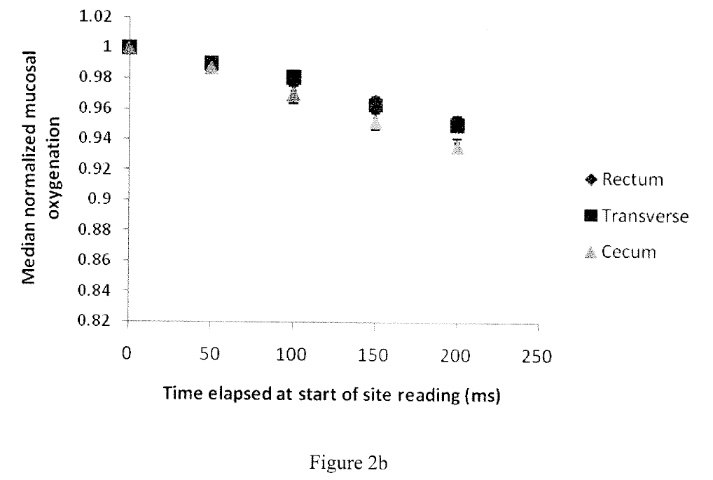

Based upon the EIBS measurements, those measurements can be used to obtain the estimated PLS and the estimated OHb, to obtain an indication on the healthiness of the entire colon. Variants of the estimated PLS and the estimated OHb can also be used to obtain this indication, such as a measurement of change in OHb over time (see, for example, the change when the diffusion is occurring as shown in FIG. 2 below), such that one can monitor the rate of change over a period such as 100 ms and see if greater than the normal change during that timeframe results) as the indication or a measure of a ratio of one blood vessel diameter at one depth to another blood vessel diameter at another depth as the indication.

### Blood Vessel Size Calculation

The following discussion pertains to calculating effective blood vessel size of superficial tissue. The same parameter is also referred to as the hemoglobin (Hb) packaging length scale (PLS). PLS is measured using a polarization-gated probe. In a particular embodiment, this polarization includes three 200 Mm-core diameter multimode fibers, one of which was used as an illumination channel while the others were used for light collection. The illumination fiber was coupled to a broadband light source. Two thin film polarizers were mounted onto the proximal tip of the probe to polarize the incident light and to enable collections of co-polarized, I_{∥}(λ), and cross-polarized, I_{⊥}(λ), scattering signals. A graded refractive index (GRIN) lens attached to the fiber tip served to collimate light from the illumination fiber as well as focus backscattered light from the sample into the two collection fibers. The GRIN lens also ensured that the collection fibers received scattered light from the same area (spot diameter=0.7 mm) that the illumination fiber illuminated. The tip of the GRIN lens was polished at an 8° angle to prevent specular reflection. At the distal end of the probe, the two collection fibers were coupled to a spectrometer which recorded the spectra of light returned from tissue between 450-700 nm. While a near-continuous spectra of light is preferable, at least three discrete wavelengths that include at least one wavelength each of high hemoglobin absorption, moderate hemoglobin absorption, and low hemoglobin absorption are needed. This particular polarization gating probe collects reflectance signals from three penetration depths that correspond, in this embodiment to a SHALLOWEST=CoPol−CrossPol; MEDIUM=CoPol only; and DEEPEST=CrossPol ONLY). Alternatively, other configurations are possible. For example, for a probe with only 2 collection fibers, an illumination fiber and a single polarized (either CoPol or CrossPol) receive fiber, then only 1 penetration depth is required.

In this regard, it is noted that PLS, and preferably both PLS and OHb, obtained from a single depth can provide sufficient diagnostic information, though having this information obtained from multiple depths, particularly multiple depths within the mucosal layer, can provide for even better results since different tissue depths may have different diagnostic sensitivities. It is also noted that a plurality of depths can be obtained in one measurement with EIBS by looking at co-pol and cross-pol and co-pol minus cross-pol received signals.

The collection fibers of the probe obtain signals that are co-polarized (I_{∥}) and cross-polarized (I_{⊥}) with respect to the incident polarization direction. Since the multiple scattering of light randomizes its polarization direction, the I_{⊥}-channel exclusively samples multiple-scattered light, while the I_{∥} channel samples the combination of short-traveled light and the multiply scattered light (I_{∥} and I_{⊥} collect the same amount of depolarized light). Thus, the difference between these two signals, after normalization by the collection efficiency of each channel (ΔI), isolates the shortest-traveled light. In order to minimize system effects from ambient background light as well as varying fiber coupling efficiencies, we used the following normalization scheme:

$$\begin{matrix}{I}_{⊥} ( λ ) = \frac{{i}_{⊥} ( λ ) - {BG}_{⊥} ( λ )}{K \cdot {RF}_{⊥} ( λ )} & ( 1 ) \\ {I}_{∥} ( λ ) = \frac{{i}_{∥} ( λ ) - {BG}_{∥} ( λ )}{{RF}_{∥} ( λ )} & ( 2 ) \\ Δ I ( λ ) = {I}_{∥} ( λ ) - {I}_{⊥} ( λ ) & ( 3 )\end{matrix}$$

Where I_{⊥}(λ), I_{∥}(λ), and ΔI(λ) represent cross-polarization, co-polarization, and differential-polarization signals after normalization, respectively. i represents the measured signal when the probe is in contact with a sample, BG represents the background signal obtained when the probe tip is in contact with water, RF represents the signal obtained from a polytetrafluoroethylene reflectance standard, and K is a constant that represents the effectiveness of the reflectance standard at depolarizing light. The constant K was determined to be 0.89 for a specific white standard used. In general this constant K is an experimentally determined ratio of cross-polarized to co-polarized received light when illuminated with a linearly polarized source.

Thus, signals from three penetration depths were calculated by utilizing two independent measurements from orthogonally polarized collection channels. Note that although the I_{⊥}(λ) signal corresponds to the longest penetration depth of the three, this signal is still superficial compared to the diffusion regime of photon scattering. The signals from the three different penetration depths described above can be analyzed individually for Oxy and DeOxy hemoglobin and effective blood vessel size as described below. Doing so yields estimates of the OHb, DHb, and PLS parameters for three different tissue depths which may have different diagnostic sensitivities. For example, surface OHb (derived from the spectrum from equation 3 above), is highly diagnostic.

### Quantifying Effective Blood Vessel Size

Quantification of the concentrations of oxygenated and deoxygenated hemoglobin in tissue has been previously disclosed. Briefly, we developed an algorithm based on the Beer-Lambert law. The model assumes that the variability in path length due to differences in optical properties within the sample is small for each of the three types of polarization gated signals.

In this particular application, as related to PLS determination, it is noted that the extinction coefficients (Aohb and Adhb listed below) are changed with PLS, and calculating them with PLS not only gives an estimate of PLS but also results in a more accurate OHb and DHb reading. The attenuation due to absorption has an inverse exponential relationship with the absorber concentration and the spectrum of light returned from tissue can be approximated as follows:

I(λ)=I_{scattering}(λ)·e^{-α}^{_{OHb}}^{·A}^{_{OHb}}^{(λ)-α}^{_{DHb}}^{·A}^{_{DHb}}^{(λ)}, (1)

where I_{scattering}(λ) is the scattering signal from the sample that would be observed, if it were devoid of absorbers, A_{OHb}(λ) is the absorption spectrum of oxy-hemoglobin, A_{DHb}(λ) is the absorption spectrum of deoxy-hemoglobin. α_{OHb} and α_{DHb} are the products of light path length and the concentrations of the oxygenated and deoxygenated forms of hemoglobin, respectively. The absorption spectra of HbO₂ and Hb, compiled from published sources.

In the absence of blood supply (Hb concentration=0), I(λ)=I_{scattering}(λ). If Hb concentration is not zero, the recorded spectrum is altered due to the presence of Hb absorption bands. This allows for quantification of oxy and deoxy-Hb concentrations. The fact that Hb is not distributed uniformly throughout the tissue volume but instead packed within red blood cells (RBC) and RBCs are in turn concentrated within blood vessels further alters the spectra. This “Hb-packing” phenomenon enables quantification of effective blood vessel size (PLS) and further improves the accuracy of oxy and deoxy-Hb concentration measures.

In order to measure PLS, the absorption spectra have to be corrected for hemoglobin packing following methods described by J. C. Finlay, and T. H. Foster, “Effect of pigment packaging on diffuse reflectance spectroscopy of samples containing red blood cells,” Opt Lett 29, 965-967 (2004). When hemoglobin is confined or packed into erythrocytes and blood vessels, hemoglobin molecules within the same erythrocyte may shield each other from the incident light in the same way as erythrocytes within a blood vessel may also shield each other from incident light. Additionally, the volume of the sample not occupied by erythrocytes provides possible light paths that do not sample any hemoglobin. The end result is a flattening of the absorption spectra for both oxy-Hb and deoxy-Hb. The corrected extinction spectra, A(λ), can be found by multiplying the extinction spectrum in solution by a distortion coefficient described by Finlay and Foster referenced above. For example, the extinction spectra for deoxy-Hb (DHb) is obtained as

$${A}^{DHb} ( λ ) = {A}_{solution}^{DHb} ( λ ) ⋆ [ \begin{matrix}( 1 - ( \begin{matrix}\frac{2}{{( 2 {μ}_{a}^{DHb} ( λ ) R )}^{2}} ( 1 - ( 2 {μ}_{a}^{DHb} ( λ ) R + 1 ) \\ exp ( - 2 {μ}_{a}^{DHb} ( λ ) R )\end{matrix} ) ) \\ \frac{3}{4 R {μ}_{a}^{DHb} ( λ )}\end{matrix} ] , \qquad ( 2 )$$

where μₐ^{Hb} is the absorption coefficient of DHb in a single erythrocyte and R is the packing length scale of the DHb. Here μₐ^{DHb} is equal to Aₛₒₗᵤₜᵢₒₙ^{DHb}(λ)*[DHb] where [DHb], the concentration of DHb in a single erythrocyte, was determined to be 6.25 mM for a suspension of deoxygenated red blood cells. An analogous equation also applies for the corrected absorption spectrum of OHb. For a spectrum measured from a solution of erythrocytes, R corresponds to the radius of a red blood cell. However, when erythrocytes are further packed into blood vessels, the packaging effect is no longer due to the cells themselves and instead becomes a measure of Hb packing as seen by all possible light paths through a blood vessel. Thus, R, the length scale of the packed red blood cells inside a blood vessel, is referred to as an effective blood vessel size.

Equations 1 and 2 show a spectrum recorded from tissue is related to parameters α_{OHb}, α_{DHb}, and R. Now we discuss how these parameters and, in particular, how effective blood vessel size R, can be determined from a tissue spectrum. Effective blood vessel size is determined as part of the algorithm previously developed to quantify oxy and deoxy Hb concentration. Knowledge of the “endogenous” scattering spectrum, I_{scattering}(λ) would allow us to apply Eqs. 1-2 and deduce α_{OHb} and α_{DHb} which best fit the measured spectra, I(λ). Although the exact functional form of the scattering spectrum is not known a priori, oxy and deoxy Hb concentration can still be estimated given the fact that I_{scattering}(λ) is expected to be a slowly-varying function of wavelength and should not exhibit oxy and deoxy Hb absorption bands.

To implement the algorithm set forth by Equations (1) and (2) above, we assumed the scattering spectrum to be in the form of the Born approximation for the reduced scattering coefficient for a random medium with continuous refractive index fluctuations with large correlation length scale, which has the following form:

I_{scattering}(λ)∝λ^{2β-4}, (3)

where β is the parameter characterizing the type of the refractive index correlation function (0<β<2). For a given tissue site, the probe measures spectrum I(λ). I_{scattering}_{_{—}}_{measured}(λ) is then calculated by applying equations 1-2 for a given combination of parameters α_{OHb}, α_{DHb}, and R:

I_{scattering}_{_{—}}_{measured}(λ)=I(λ)·exp[α_{DHb}·A_{DHb}(λ)+α_{OHb}·A_{OHb}(λ)]. (4)

This is the scattering spectrum that would be observed if the parameters α_{OHb}, α_{DHb}, and R correctly characterized tissue microvasculature. If the choice of the parameters is indeed correct, I_{scattering}_{_{—}}_{measured}(λ)∝I_{scattering}(λ)∝λ^{2β-4}. On the other hand, if the choice is incorrect, I_{scattering}_{_{—}}_{measured}(λ) would still exhibits Hb absorption features either as Hb absorption “deeps” (if α_{OHb}, α_{DHb} underestimate the true concentrations) or Hb “humps” (if α_{OHb}, α_{DHb} overestimate the true concentrations). Therefore, the coefficients α_{OHb}, α_{DHb}, and R, and β are chosen such that the sum of square error between I_{scattering}_{_{—}}_{measured}(λ) and λ^{2β-4} is minimized. This can be accomplished by a variety of optimization algorithms.

Calculation of PLS imposes an additional requirement on the spectrum of illuminated and collected light as compared to the oxy and deoxy hemoglobin calculations previously discussed: a broad wavelength range is important to measure PLS. In particular, it is imperative that this wavelength range includes wavelengths for which oxy and deoxy Hb absorption is negligible. For example, 480-680 nm wavelength range is adequate to calculate the effective blood vessel size. If the wavelength range does not include wavelengths for which oxy and deoxy Hb absorption is negligible, PLS calculation becomes is inaccurate and unstable. In this case, although oxy and deoxy Hb concentrations may still be determined, even a small deviation in signal (e.g. due to noise) may result in a disproportionate deviation in the calculated value of PLS. This is in part because the optimized function has a number of similar local minima in the functional space. For example, although the range from 450 nm up to 600 nm may be sufficient to estimate Hb concentrations, it is insufficient to determine PLS because it does not contain a range of wavelengths that exhibit low hemoglobin absorption.

### Prediction Rules Based Upon PLS and OHb

The PLS and OHb parameters of EIBS can effectively be used, either singularly, or preferably together as a screening test for colon cancer, and this is confirmed by the Supporting Data provided below. Before providing this discussion, however, a discussion of prediction rules is discussed.

Identification of Patients with Advanced Adenomas vs. Control Patients Based on Rectal EIBS.

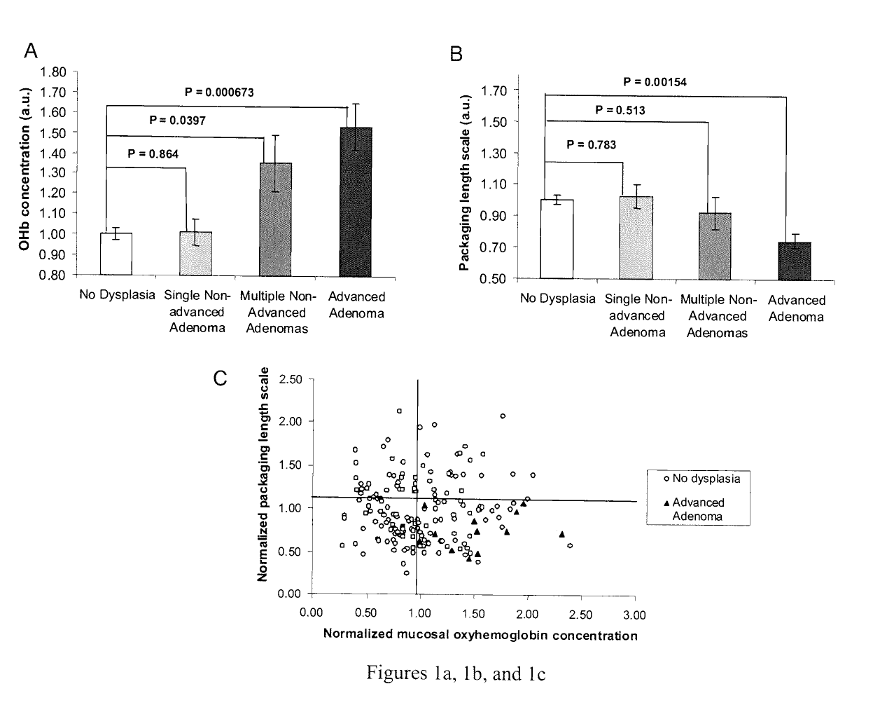

To differentiate patients with and without adenomas based on the analysis of rectal mucosal microvasculature, a prediction rule can be developed based on the two EIBS parameters discussed above: oxy-Hb concentration and PLS, the effective blood vessel size. It is also clear from the scatter plot shown in FIG. 1(c) that OHb and PLS are uncorrelated (Pearson r-value=0.0456) indicating that they are independent predictors of neoplasia risk. For example, a prediction rule can be designed as follows. First, a threshold is determined for OHb based on the receiver observer characteristics (ROC) curve to obtain a desirable sensitivity and specificity. For example, a threshold value of OHb (defined here as OHb_t) can be chosen so that sensitivity=100%. A threshold for the effective blood vessel size (EBVS_t) is obtained based on similar considerations. Each selected threshold could then be used independently as a simple screening test for colon cancer. For example, at risk patients would be those with a normalized oxyhemoglobin value greater than OHb_t in one test. Separately, a second test would classify patients at risk if they have a normalized packaging length scale less than EBVS_t. While each of the above rules can be used separately, better results are achieved based upon a further combined prediction rule made by classifying a patient as positive if and only if the patient has an effective blood vessel size below EBVS_t and OHb value above OHb_t. This rule yields 100% sensitivity and 74% specificity. After leave-one-out cross-validation, the sensitivity remained 100% with 71% specificity. It is noted that the obtained normalized oxyhemoglobin value and obtained normalized packaging length scale are preferably obtained from a control group of healthy individuals.

The data supporting the above screening prediction rule discussion is now provided.

Supporting Data. 216 patients comprising 165 who were adenoma-free, 39 with single non-advanced adenomas, 9 with multiple non-advanced adenomas and 12 with advanced adenomas were studied. Patients undergoing colonoscopy had, on average, 10 readings taken using a fiber-optic EIBS probe in the endoscopically normal rectum. Our analysis showed that superficial (<100 μm) OHb was altered in subjects harboring either multiple non-advanced adenomas or single advanced adenomas. As seen in FIG. 1(a), there was a step-wise progression that paralleled the neoplastic risk. Rectal PLS which was decreased in advanced adenoma patients (FIG. 1(b)). When the PLS and OHb were combined into a simple prediction rule, advanced adenoma patients were clearly segregated (FIG. 1(c)). The area under the receiver operator characteristic curve (AUROC) for the prediction rule based on rectal OHb and PLS was excellent, 0.927.

This data shows that the screening test for colon cancer based on EIBS is effective. Thus, a patient with a negative rectal EIBS test can forego colonoscopy, whereas a patient with a positive rectal EIBS test would need colonoscopy. Since without this pre-screen, all patients would need colonoscopy, a modest false positive rate is acceptable whereas false negatives would potentially result in a clinically poor outcome. Therefore, a threshold can be set to achieve sensitivity of 100% and specificity was 75%. Confounding by demographic factors including age and smoking history and confounding by benign lesions including hemorrhoids, diverticulosis and benign hyperplastic polyps have been determined to have no significant effect on the screening test outcome.

The screening test described above can be used as a way to target only those patients who are most likely to harbor neoplasia. This in many ways is the basis for using fecal occult blood test (FOBT) or flexible sigmoidoscopy as primary screening tests and sending patients to colonoscopy only if these are positive. The problem is that the sensitivity of these existing tests is remarkably low (FOBT has ˜10% sensitivity for advanced adenomas). For a pre-screen test to be valuable it would have to have an outstanding sensitivity for clinically significant lesions (advanced adenomas or carcinomas). Specificity should be good but does not have to be perfect given the tolerance for false positives (which would obligate colonoscopy, however without the test everyone should be getting the colonoscopy).

Thus the rectal EIBS screening test has at least the following applications:

1) Rectal EIBS screening test as a stand-alone test during an annual physical exam by a primary care physician or a gynecologist (in females). This rectal EIBS screening test can be performed without the need for colonoscopy or colonic preparation. The latter is one of the major reasons for patients' non-compliance. Initially, the rectal EIBS screening test may be performed on patients who refuse colonoscopy. Based on the results of the rectal EIBS screening test, a patient may be indicated to receive a colonoscopy (which he will be more compliant to given the rectal EIBS screening test result). Thus, patients at a higher risk for CRC will receive colonoscopies as appropriate, whereas low-risk patients will not undergo these expensive, uncomfortable procedures.

2) Rectal EIBS screening test during flexible sigmoidoscopy (FS) (endoscopic evaluation of the distal colon). FS has been used for CRC screening for the last several decades. FS examines only the distal part of the colon. If an adenoma is identified, a patient undergoes full colonoscopy for both distal polyp removal and identification of the possible proximal lesions. Patient compliance is better because of less discomfort and, equally importantly, a more tolerable colonic purge. From a societal perspective, flexible sigmoidoscopy's advantages include that it is relatively inexpensive, has a lower complication rate and can be performed by the primary care physicians or even nurse practitioners (thus increasing endoscopic capacity). The criticism of flexible sigmoidoscopy has centered around its inability to assess for lesions in the proximal colon which has resulted in it being largely eschewed by colonoscopy. This limitation is particularly important in women given their higher prevalence of isolated proximal neoplasia. Indeed, flexible sigmoidoscopy identified two-thirds of the men with advanced neoplasia (advanced adenomas or carcinomas) but only one-third of women. A rectal EIBS screening test during flexible sigmoidoscopy either by a PCP or a nurse-practitioner can help identify those patients with proximal neoplasia but no distal adenomas.

Acquisition of On Contact Measurements. When a probe is brought in contact with tissue, the probe exerts a pressure onto the tissue. This contact reduces blood flow. While oxygen continues to diffuse from the arterial red blood cells into the tissue, it is expected that the concentration of oxy-Hb measured by the probe should go down with time. We tested this hypothesis by recording 5 consecutive readings 50 msec each. FIG. 2 shows that the measured oxy-Hb concentration as well as tissue oxygenation decreases with time, supporting the hypothesis. The decrease is expected to be an exponential function of time. This effect has two important implications:

1) Unless it is known when the probe gets in contact with the tissue, there will always be a finite delay between the contact and the point-in-time when the signal acquisition is initiated by the operator (e.g. endoscopist). This delay introduces an uncertainty and extra variability into the values of oxy-Hb concentration measured by the probe. As illustrated in FIG. 2, this potential variability can be quite significant: a delay as small as 0.25 sec can result in an apparently lower oxy-Hb concentration by at least 15-20%. Thus, in order to reduce variability and ensure accurate measurements, it is imperative to be able to determine the time of the probe-tissue contact.

2) The effect presents an opportunity to measure the rate of oxygen diffusion, which in turn is indicative of the metabolic rate of the tissue. This can serve as yet another marker of physiological and pathophysiological processes. On contact measurements can be achieved by a number of approaches. One approach is based on the fact that the scattered light intensity collected by the probe is inversely related to the probe-to-tissue distance and vanishes quickly when the probe-to-tissue distance exceeds a critical distance. For example, for the probe used to collect the data discussed above, this critical distance is <1 mm. In one implementation, the intensity of light collected by the probe is being continuously monitored. A rapid increase of the intensity beyond the critical level indicates that the probe is in contact with the tissue. This rapid increase can be used to automatically trigger acquiring the signal that is used as the correct patient data. The continuous monitoring does not have to be performed for the entire spectrum. In order to save both signal acquisition and analysis time, it is sufficient to record and analyze signal intensity for a narrow band of wavelengths. For example, one could look at the reflected intensity of a single or narrow range of wavelengths, and once the intensity surpasses a threshold, one would know the probe is in contact with tissue. In this approach, the monitoring may be performed every 50 msec or less.

Obtaining measurements can thus occur at the time of contact, as well as after a delay period after contact, and, preferably both at and after the time of contact.

Further, multiple different locations of the distal colon can be tested, with the results then averaged together to provide the screening indication, Though as few as one measurement can be made, having between 3-6 measurements is preferred, with there being little advantage to having more than 10 measurements. Also, the screening test herein can also be used to decide when to perform another test to re-determine whether the living tissue within the organ may be abnormal, based upon the reading determined. Thus, the closer that the estimated blood vessel size and estimated OHb is to the normalized values, the sooner the physician may suggest that the patient return for another screening test.

The foregoing description of the exemplary embodiments of the invention has been presented only for the purposes of illustration and description and is not intended to be exhaustive or to limit the invention to the precise forms disclosed. Many modifications and variations are possible in light of the above teachings.

What is claimed:

1. A method of providing an indication that living tissue within an entire colon of a human body may be abnormal comprising the steps of:
inserting a probe such that a light source within the probe is disposed in a location that is at an inner surface of a distal part of the colon;
illuminating, at the location, tissue of the inner surface of the distal part of the colon and microvasculature within a mucosal layer therein with light from the light source that is emitted from the probe, wherein the tissue that is illuminated with the light does not contain the living tissue that may be abnormal;
detecting interacted light that results from the step of illuminating the tissue as detected data using the probe, wherein the interacted light is obtained substantially from the light that then interacts with blood in the microvasculature of the mucosal layer that is within the tissue of the distal part of the colon, which tissue does not contain the living tissue that may be abnormal;
estimating effective blood vessel size in the microvasculature using the detected data; and
obtaining the indication that the living tissue within the entire colon may be abnormal using the estimated effective blood vessel size.

2. The method according to claim 1 wherein the step of estimating also estimates oxygenated hemoglobin, and wherein the step of obtaining the indication uses both the estimated oxygenated hemoglobin and the estimated blood vessel size.

3. The method according to claim 2 wherein the estimated oxygenated hemoglobin and the estimated blood vessel size are compared against an oxygenated hemoglobin threshold and an estimated blood vessel size threshold, such that the indication that the living tissue within the colon may be abnormal results if and only the estimated blood vessel size is below the estimated blood vessel size threshold and the estimated oxygenated hemoglobin is above the oxygenated hemoglobin threshold.

4. The method according to claim 3 wherein the oxygenated hemoglobin threshold and the estimated blood vessel size threshold are obtained from averages of measurements obtained from a control group of healthy individuals.

5. The method according to claim 4 further including the step of performing a colonoscopy if the indication is that the living tissue may be abnormal.

6. The method according to claim 2 wherein the step of estimating the oxygenated hemoglobin is recalculated using the estimated blood vessel size.

7. The method according to claim 2 wherein a plurality of the estimated oxygenated hemoglobin are obtained from over a period of time, and wherein the step of obtaining the indication includes obtaining a rate of change of estimated hemoglobin using the plurality of the estimated oxygenated hemoglobin.

8. The method according to claim 1 wherein the step of detecting takes place immediately upon contact of the probe with the tissue.

9. The method according to claim 1 wherein the step of detecting takes place a delay period after contact of the probe with the tissue.

10. The method according to claim 1 wherein the step of detecting takes place both immediately upon contact of the probe with the tissue and a delay period after contact of the probe with the tissue.

11. The method according to claim 1 wherein a plurality of the estimated blood vessel sizes are obtained from multiple mucosal depths.

12. The method according to claim 11 wherein a ratio of the estimated blood vessel sizes from different ones of the multiple mucosal depths is used to provide the indication.

13. The method according to claim 1 wherein the step of detecting detects at least one of the following components of the interacted light: co-polarized, cross-polarized, and unpolarized interacted light.

14. The method according to claim 13 wherein the step of detecting detects interacted light at a plurality of penetration depths between a top of the inner surface to a submucosal layer.

15. The method according to claim 13 wherein the step of detecting the tissue detects interacted light at a plurality of penetration depths between a top of the inner surface to the mucosal layer.

16. The method according to claim 1 wherein the steps of inserting, illuminating and detecting are performed using a probe disposed at least partially within an endoscopic device.

17. The method according to claim 1 wherein the step of obtaining the indication includes the step of comparing the estimated blood vessel size with a baseline vessel size.

18. The method according to claim 17 further including the step of establishing the baseline vessel size.

19. The method according to claim 18 further including the step of establishing the baseline blood vessel size based upon measurements of blood vessel size of a plurality of human bodies other than the human body.

20. The method according to claim 1 wherein the indication from the step of obtaining indicates that the living tissue may be abnormal at a future point in time.

21. The method according to claim 1 further including the step of using the indication to decide when to perform another test to re-determine whether the living tissue within the distal colon may be abnormal.

22. The method according to claim 1 wherein the illuminated tissue is at least one of histologically normal, macroscopically normal, and endoscopically normal.

23. The method according to claim 1 the probe is inserted into the distal colon without any prior colon purging.
